(19) United States
(12) Patent Application Publication
(10) Pub. No.: US 2019/0002360 A1
(43) Pub. Date: Jan. 3, 2019

(54) Organic Liquid Having High Nitrate Content for Use in the Organic Cultivation of Plants on a Substrate

(71) Applicant: Ven der Knaap Diensten B.V., Kwintsheul (NL)

(72) Inventors: Carolus Wilhelmus Maria de Bruijn, Monster (NL); Bartholomeus Cornelis 't Hoen, Monster (NL)

(73) Assignee: Ven der Knaap Diensten B.V., Kwintsheul (NL)

(21) Appl. No.: 15/748,605

(22) PCT Filed: Aug. 2, 2016
(86) PCT No.: PCT/EP2016/068446

§ 371 Date: Jan. 29, 2018

(30) Foreign Application Priority Data
Aug. 3, 2015 (NL) 2015253
Jan. 28, 2016 (NL) 2016175

(51) Int. Cl. C05F 17/00; C05F 5/00; C05F 7/00; C12P 3/00

(52) U.S. Cl. CPC C05F 17/0027; C12P 3/00; C05F 7/00; C05F 5/00

(57) ABSTRACT

The present invention relates to a method for producing an organic liquid having a high nitrate content suitable for organically cultivating plants on a substrate, the organic liquid or plant nutrition thus produced, and use of the organic liquid or plant nutrition in a method for organically producing plants on a substrate. The method comprises a) contacting a solution comprising proteins and/or amino acids as a source of organic nitrogen and a liquid with ammonification bacteria to convert organic nitrogen into ammonium (NH₄⁺) and b) contacting the solution with nitrifying bacteria to convert ammonium (NH₄⁺) to nitrate (NO₃⁻).

The present invention relates to the cultivation of plants on a substrate, and in particular to the organic cultivation of plants on a substrate.

Plants require several nutrients to grow, such as nitrogen (N), phosphorus (P), potassium (K), calcium (Ca), sulphur (S), magnesium (Mg), boron (B), chlorine (Cl), manganese (Mn), iron (Fe), zinc (Zn), copper (Cu), and molybdenum (Mo). The nitrogen as present in the atmosphere and as proteins and/or amino acids in plant and animal matter is, however, not available to plants. For nitrogen to be available for uptake by plants it needs to be present in the form of ammonium (NH₄⁺), nitrate (NO₃⁻) or urea (NH₂). Microorganisms, such as fungi and bacteria present in the soil, can convert proteins and amino acids into nitrate, as is shown in FIG. 1.

To provide plants with the optimum growing conditions, plant growers often use fertilizers. These fertilizers comprise nitrogen compounds, such as proteins, amino acids, and/or ammonium. Once applied to the soil, these compounds are converted to nitrate by the process as described above.

In greenhouses, plants are often grown on a substrate. This is advantageous as the growing conditions can be fully controlled and it is easy to disinfect the substrate for subsequent use. Furthermore, the incidence of soil-borne diseases and pests, such as for instance Fusarium phytopthora, is greatly reduced. A substrate can be composed of a variety of different materials such as stone wool, peatmoss, coco, and/or perlite.

Proteins and/or amino acids present in, or added to, the substrates are converted into the nitrate required for plant growth. This conversion is achieved by microorganisms present in, or added to, the substrate. However, the nitrate that can be made available solely by the microorganisms in the substrate is insufficient for decent plant growth. This is likely due to the limited conversion rate of the microorganisms in the substrate.

This problem may be solved by increasing the volume of the substrate, however, this is undesired as this will result in too much vegetative growth and therefore in a non profitable yield. The best way in providing the growing plants with sufficient nitrate would be adding nitrate to the substrate, for instance as part of a fertilizer.

It would even be more advantageous if plants on substrates can be grown organically, as organic products have a better quality, and organic vegetables and fruits also have a better taste. This is reflected by the increasing demand for organic products on the market.

It has been tried to produce plants on substrates which are 100% organic, however, this still leads to unsatisfactory results. Therefore, minimal amounts of inorganic fertilizers are still being used. The disadvantage of this is that the plants produced with use of inorganic fertilizers cannot be qualified as being organically produced and cannot be labelled and sold as such.

The currently existing organic fertilizers do not contain any nitrate and are therefore not suitable for the organic cultivation of plants on a substrate.

“Organic” in the context of the present invention means that the materials used are all derived from plant and/or animal matter. In the organic cultivation of plants, it is also accepted if a material comprises certain natural minerals not derived from plant or animal matter, such as for instance magnesium sulphate and trace elements. However, nitrogen is not one of these. At present, no plant nutrition exists that comprises nitrate derived from plant and/or animal matter. There is thus no plant nutrition comprising nitrate that is fully organic.

It is therefore an object of the present invention to provide organic plant nutrition comprising nitrate, which plant nutrition is suitable for growing plants on a substrate.

In the research leading to the present invention, the inventors found that a liquid with a high nitrate content could be produced by the ammonification and nitrification of proteins and/or amino acids in a one step batch reaction, as well as in a two steps continuous flow-through system.

In the one step batch process, both ammonification bacteria and nitrifying bacteria are brought into contact with a solution comprising proteins and/or amino acids at the same time. The proteins and/or amino acids are converted into ammonia (NH₃) and subsequently into ammonium (NH₄⁺) by the ammonification bacteria, and the ammonium (NH₄⁺) is converted into nitrite (NO₂⁻) and subsequently into nitrate (NO₃⁻) by the nitrifying bacteria. These two conversion steps occur in parallel, since both ammonification bacteria and nitrifying bacteria are present.

“At the same time” means that the solution comprises both types of bacteria at a particular time. The bacteria do thus not have to be added simultaneously but may also be added subsequent to each other.

The invention thus relates to a method for producing an organic liquid having a high nitrate content, comprising:
a) contacting a solution comprising proteins and/or amino acids as a source of organic nitrogen and a liquid with ammonification bacteria to convert organic nitrogen into ammonium (NH₄⁺); and
b) contacting the solution with nitrifying bacteria to convert ammonium (NH₄⁺) to nitrate (NO₃⁻).

In the two steps continuous flow-through system, the solution comprising proteins and/or amino acids is first brought into contact with the ammonification bacteria and subsequently with the nitrifying bacteria. In this case, the nitrate is produced in two steps, wherein in a first step the proteins and/or amino acids are converted into ammonia (NH₃) and subsequently into ammonium (NH₄⁺), and the ammonium (NH₄⁺) is converted into nitrite (NO₂⁻) and subsequently into nitrate (NO₃⁻) in a second step. The two steps take place in separate containers. Because the ammonification and nitrification are separated into two steps, no stagnation of the nitrate production takes place. This enables a continuous process for the production of an organic liquid having a high nitrate content, which cannot be achieved when ammonification and nitrification are performed in a single step, such as in a batch process. This safes time, reduces labour and ensures the continuous availability of an organic liquid having a high nitrate content for plant irrigation.

The invention thus also relates to a method for producing an organic liquid having a high nitrate content, comprising:
a) contacting a solution comprising proteins and/or amino acids as a source of organic nitrogen and a liquid with ammonification bacteria to convert organic nitrogen into ammonium (NH₄⁺); and
b) subsequently contacting the solution with nitrifying bacteria to convert ammonium (NH₄⁺) to nitrate (NO₃⁻).

The solution resulting from these methods of the invention is an organic liquid having a high nitrate content. The invention thus also relates to an organic liquid having a high nitrate content obtainable by the method of the present invention. This organic liquid having a high nitrate content is also referred to herein as “the organic liquid” or “the organic liquid of the invention”. The organic liquid may be a fertilizer, or be part of a fertilizer. The terms “fertilizer” and “plant nutrition” relate to the same and are used interchangeably herein.

The organic liquid of the invention can be used to irrigate plants grown on substrate. By using the method of the present invention, proteins and amino acids are converted into nitrate which is then added to the substrate. As a result, the plants have access to a sufficient amount of nitrate and no inorganic material is being used.

The organic liquid obtained by the methods of the present invention is suitable for organically cultivating plants on a substrate. The invention thus also relates to a method for producing an organic liquid having a high nitrate content, comprising:
a) contacting a solution comprising proteins and/or amino acids as a source of organic nitrogen and a liquid with ammonification bacteria to convert organic nitrogen into ammonium (NH₄⁺); and
b) contacting the solution with nitrifying bacteria to convert ammonium (NH₄⁺) to nitrate (NO₃⁻), thereby obtaining an organic liquid having a high nitrate content,

wherein the organic liquid having a high nitrate content is suitable for organically cultivating plants on a substrate.

The present invention also relates to a method for producing an organic liquid having a high nitrate content, comprising:
a) contacting a solution comprising proteins and/or amino acids as a source of organic nitrogen and a liquid with ammonification bacteria to convert organic nitrogen into ammonium (NH₄⁺); and
b) subsequently contacting the solution with nitrifying bacteria to convert ammonium (NH₄⁺) to nitrate (NO₃⁻), thereby obtaining an organic liquid having a high nitrate content,

wherein the organic liquid having a high nitrate content is suitable for organically cultivating plants on a substrate.

With “high nitrate content” is meant that when a sample is taken and contacted with a nitrate test strip, the nitrate test strip turns into a specific colour indicated by the manufacture to be indicative of a high nitrate content. When using the 110020 Nitrate Test, a colorimetric method with test strips for 10-25-50-100-250-500 mg/l NO₃⁻ (MQuant™), purple is indicative of a high nitrate content. A person skilled in the art knows that several different nitrate test strips are available that may be used and is familiar with how to use them. The exact nitrate concentration can also be quantified by a certified laboratory, following methods known to a person skilled in the art. A nitrate concentration of at least 4 mmol/l is considered as a high nitrate content. This content is sufficient for the growth of a plant.

In one embodiment, the organic liquid produced has a nitrate content of at least 4 mmol/l. Preferably, the organic liquid produced has a nitrate content in the range of 4-30 mmol/l, more preferably in the range of 4-25 mmol/l, even more preferably in the range of 4-20 mmol/l, even more preferably in the range of 4-15 mmol/l, even more preferably in the range of 4-10 mmol/l, even more preferably in the range of 8-30 mmol/l, even more preferably in the range of 8-25 mmol/l, even more preferably in the range of 8-20 mmol/l, even more preferably in the range of 8-15 mmol/l, and most preferably in the range of 8-10 mmol/l.

The organic substance used in step (a) of the above methods may be any organic substance comprising nitrogen as part of a biological compound. The biological compound may for instance be ammonium (NH₄⁺), proteins and/or amino acids. Preferably, an organic substance is used which has a high content of ammonium (NH₄⁺), proteins and/or amino acids. The inventors obtained 8-10 mmol/l nitrate with an organic substance comprising about 247 g nitrogen (N). At least 4 mmol/l nitrate may be obtained with an organic substance comprising at least 100 g nitrogen (N). The organic substrate may also be enriched solely with ammonium (NH₄⁺), proteins and/or amino acids. For instance, commercially available plant nutrition, such as organic products from Plant Health Cure or Koppert Biological systems, may be used. The organic substance may also comprise the minerals not derived from plant and/or animal matter which are accepted by the regulations to be part of an organic substance, such as for instance magnesium sulphate and trace elements. It is however preferred that the organic substance is free of these minerals.

The liquid in which the protein and/or amino acid-rich substance is dissolved is preferably water or an aqueous solution. The solution comprising the organic proteins and/or amino acids dissolved in a liquid is also referred to herein as influent.

It is important that the electric conductivity (EC) of the influent does not exceed 2.0 mS/cm, as this may block nitrate production. It is therefore preferred that the solution comprising proteins and/or amino acids of step (a) has an EC of below 2.0 mS/cm.

The bacteria employed in step (a) may be any bacteria capable of converting proteins and/or amino acids into ammonia (NH₃) and ammonium (NH₄⁺). This conversion is also referred to as ammonification or mineralization. Suitable bacteria are known to a person skilled in the art and are commercially available.

The pH of the solution in step (a) shall be between 7.0 and 9.0, and preferably between 7.5 and 9.0. For achieving optimal results, the pH is preferably in the range of 8.0-9.0, and more preferably in the range of 8.5-9.0. Thus, in a preferred embodiment, step (a) is performed at a pH of above 8.0, preferably at a pH in the range of 8.0-9.0. The desired pH may be achieved by techniques known to a person skilled in the art.

The bacteria employed in step (b) may be any bacteria capable of converting ammonium (NH₄⁺) into nitrite (NO₂⁻), and nitrite (NO₂⁻) into nitrate (NO₃⁻). These conversions are also referred to as nitrification. Suitable bacteria, such as for instance nitrifying bacteria, are known to a person skilled in the art and are commercially available.

The pH of the solution in step (b) shall be between 5.0 and 8.0, preferably between 5.5 and 8.0, and more preferably between 6.0 and 8.0. For achieving optimal results, the pH of the solution in step (b) is preferably in the range of 5.5-7.7. Thus, in a preferred embodiment step (b) is performed at a pH of between 5.0 and 8.0, preferably at a pH of between 5.5 and 7.7. The desired pH may be achieved by techniques known to a person skilled in the art.

The solution in step (a) is also referred to herein as effluent 1, and the solution in step (b) is also referred to herein as effluent 2.

The above method can be performed in a system or a device comprising a first container for dissolving the organic substance comprising proteins and/or amino acids in the liquid, a second container for ammonification, a third container for nitrification, and optionally a fourth container for receiving, storing and/or distributing the organic liquid having a high nitrate content obtained in the third container.

The ammonification and/or nitrifying bacteria may be present on biocarriers. These biocarriers may be used in the second and/or third container for increasing the surface for bacterial growth, and therefore increase the efficiency of the process.

The solution comprising the ammonification bacteria can be aerated, and the solution comprising the nitrifying bacteria must be aerated in order to improve and/or maintain bacterial life. For optimal results, both solutions should be aerated. Preferably, these solutions are aerated from below the second and/or third container.

The above method can also be performed in a system or a device comprising one or more biofilters. These biofilters may comprise the ammonification and/or nitrifying bacteria.

The invention further relates to an organic liquid having a high nitrate content. This organic liquid is suitable for organically cultivating plants on a substrate. In a preferred embodiment, the organic liquid having a high nitrate content is, or is part of, a plant nutrition.

The invention thus also relates to organic plant nutrition having a high nitrate content. This plant nutrition is suitable for organically cultivating plants on a substrate. This plant nutrition is also referred to herein as “the plant nutrition of the invention”.

In one embodiment the organic liquid or plant nutrition has a nitrate content of at least 4 mmol/l. Preferably, the organic liquid or plant nutrition has a nitrate content of between 4-30 mmol/l, more preferably in the range of 4-25 mmol/l, even more preferably in the range of 4-20 mmol/l, even more preferably in the range of 4-15 mmol/l, even more preferably in the range of 4-10 mmol/l, even more preferably in the range of 8-30 mmol/l, even more preferably in the range of 8-25 mmol/l, even more preferably in the range of 8-20 mmol/l, even more preferably in the range of 8-15 mmol/l, and most preferably in the range of 8-10 mmol/l.

This fully organic liquid or plant nutrition can be produced by converting proteins and/or amino acids into nitrate. This may be accomplished in every suitable manner known to a person skilled in the art. In a preferred embodiment, the organic liquid or plant nutrition is obtainable by the method of the present invention.

The invention further relates to the use of an organic liquid having a high nitrate content for organically cultivating plants on a substrate. The organic liquid having a high nitrate content can be used in addition to, or as part of, other organic plant nutrition. It may thus be mixed with other organic plant nutrition before adding it to the plants, or be given to the plants separately. If given separately, it may be given simultaneously, but also not simultaneously, with the other organic plant nutrition.

In a preferred embodiment the organic liquid used for organically cultivating plants on a substrate has a nitrate content of at least 4 mmol/l, more preferably of between4 and 30 mmol/l, more preferably of between 4 and 25 mmol/l, even more preferably of between 4 and 20 mmol/l, even more preferably of between 4 and 15 mmol/l, even more preferably of between 4 and 10 mmol/l, even more preferably of between 8 and 30 mmol/l, even more preferably of between 8 and 25 mmol/l, even more preferably of between 8 and 20 mmol/l, even more preferably of between 8 and 15 mmol/l, and most preferably of between 8 and 10 mmol/l. The organic liquid produced according to the present invention is and can be used as an organic fertilizer for organically cultivating plants on a substrate.

In a preferred embodiment, the organic liquid used for organically cultivating plants on a substrate is the organic liquid obtained or obtainable by the method of the present invention or the organic plant nutrition of the present invention.

The present invention further relates to a method for organically cultivating plants on a substrate, comprising adding an organic liquid having a high nitrate content to the substrate.

In a preferred embodiment the organic liquid added has a nitrate content of at least 4 mmol/l, preferably of between 4 and 30 mmol/l, more preferably of between 4 and 25 mmol/l, even more preferably of between 4 and 20 mmol/l, even more preferably of between 4 and 15 mmol/l, even more preferably of between 4 and 10 mmol/l, even more preferably of between 8 and 30 mmol/l, even more preferably of between 8 and 25 mmol/l, even more preferably of between 8 and 20 mmol/l, even more preferably of between 8 and 15 mmol/l, and most preferably of between 8 and 10 mmol/l.

In a further preferred embodiment, the organic liquid is the organic liquid obtained or obtainable by the method of the present invention or the organic plant nutrition of the present invention.

A person skilled in the art is capable of determining how much organic liquid shall be added. The organic liquid having a high nitrate content may be mixed with other organic plant nutrition and/or nutritional elements, such as for instance calcium, before adding it to the substrate, or be added to the substrate separately. If added separately, it may be added simultaneously, but also not simultaneously, with the other organic plant nutrition and/or nutritional elements.

The organic liquid having a high nitrate content may be added to the substrate by any suitable method known to a person skilled in the art.

The organic liquid having a high nitrate content may be added to any suitable substrate. A suitable substrate is an organic substrate made entirely of one or more, or a combination of, organic materials, such as for instance coco.

The plants produced on substrate may be any plant, but is in particular a plant useful in horticulture, such as vegetable plants, fruit plants, ornamental plants, including but not limited to flowers, medicinal plants, etc. In a preferred embodiment, the plant belongs to a species selected from the group consisting of Capsicum annuum, Cucumis sativus, and Solanum lycopersicum.

With the organic liquid having a high nitrate content as produced by the method of the present invention similar crop yields can be achieved as compared to crops grown when using traditional inorganic fertilizers. The organic liquid of the present invention is thus a very interesting product for the commercial organic market.

### FIGURES

FIG. 1 is a schematic overview of the nitrogen cycle.

FIGS. 2A and B are pictures of the growing pepper plants; FIG. 2C is a graph of the pepper yield.

FIGS. 3A and B are pictures of the growing tomato plants; FIG. 3C is a graph of the tomato yield.

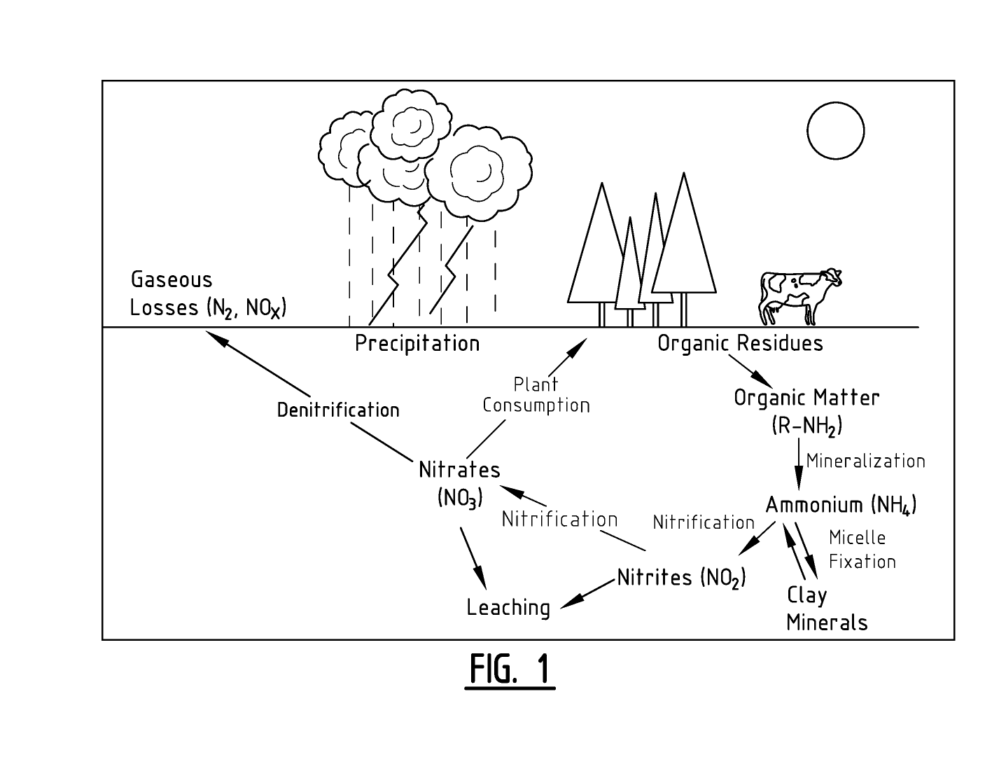

### EXAMPLE 1

### Producing a Liquid Having a High Nitrate Content

In a first container having 1000 l capacity, the influent was prepared by dissolving 5 l HOB, 0.5 l Amino 2 (Bertels B.V) and 100 ml micro nutrients (Horticoop) in 1000 l water. HOB is an internally prepared organic substance comprising proteins and amino acids. The container was aerated. The resulting influent comprised <0.2 mmol/l nitrate (NO₃⁻) and 3.3 mmol/l ammonium (NH₄⁺), and a total nitrogen (N) content of 323 mg/l. The pH was 8.2 and the EC 1.1.

The influent was transferred into a second container by a pump with a flow rate of 1000 to 2000 ml/min. This second container comprised 300 l bio carriers and was aerated. 100 ml ammonification bacteria (BactoPlus®) were added to the container to start the ammonification of proteins and/or amino acids into ammonium (NH₄⁺). The EC of the resulting solution was measured to be 2.2 and the pH was 8.4. The solution, also referred to herein as effluent 1, comprised <0.2 mmol/l nitrate (NO₃⁻) and 15 mmol/l ammonium (NH₄⁺).

The resulting effluent 1 was transferred with the same flow rate of 1000 to 2000 ml/min to a third container. This third container also comprised biocarriers. 100 ml nitrifying bacteria (BactoPlus®) were added to the container to start the nitrification of ammonium (NH₄⁺) into nitrate (NO₃⁻). The EC and pH of the resulting solution, also referred to herein as effluent 2, were measured to be 1.8 and 7.1 respectively. The solution comprised 9.1 mmol/l nitrate (NO₃⁻) and 7.2 mmol/l ammonium (NH₄⁺).

The resulting effluent 2 was transferred to a fourth container, where it was stored until use.

In other batches, nitrate (NO₃⁻) contents of 8.3 and 10.0 mmol/l were achieved.

### EXAMPLE 2

### Organic Growing of Pepper Plants

Seeds of pepper plants of the variety Marietta were sown in 100% coco substrate. Plants were cultivated as is common in the art, however, instead of watering the plants with water comprising mineral plant nutrition the plants were watered with a mixture comprising the liquid having a high nitrate content of the present invention, basin water, Horti-organic A, Horti-organic B, micro nutrients, and iron. Different mixture compositions were used throughout the experiment, starting with Mixture 1 and finishing with Mixture 4. The compositions of these mixtures are as indicated in Table 1. The mixture always comprised about 25% (volume) of the liquid having a high nitrate content of the present invention.

TABLE 1

| | Mixture 1 | | Mixture 2 | | Mixture 3 | | Mixture 4 | |
|---|---|---|---|---|---|---|---|---|
| Liquid having a | 125 | l | 150 | l | 250 | l | 250 | l |
| high nitrate | | | | | | | | |
| content of the | | | | | | | | |
| present invention | | | | | | | | |
| Basin water | 375 | l | 450 | l | 750 | l | 750 | l |
| Horti-organic A | 1.0 | l | 3.0 | l | 6.0 | l | 4.0 | l |
| Horti-organic B | 0.5 | l | 1.5 | l | 3.0 | l | 2.0 | l |
| Micro nutrients | 40.0 | ml | 50.0 | ml | 80.0 | ml | 80.0 | ml |
| Iron | 50.0 | g | 45.0 | g | 100 | g | 100 | g |

| | | | | | |
|---|---|---|---|---|---|
| Compost tea | 14.0 | l | — | — | — |

| | | | | | | | |
|---|---|---|---|---|---|---|---|
| Magnesium | — | 100 | g | 1.0 | kg | 0.25 | kg |
| sulphate | | | | | | | |

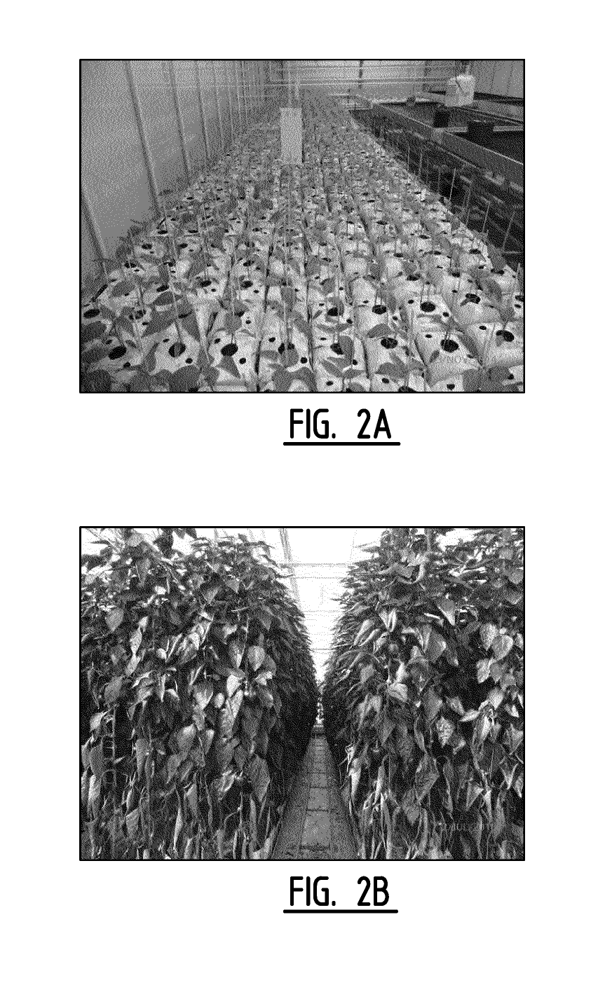

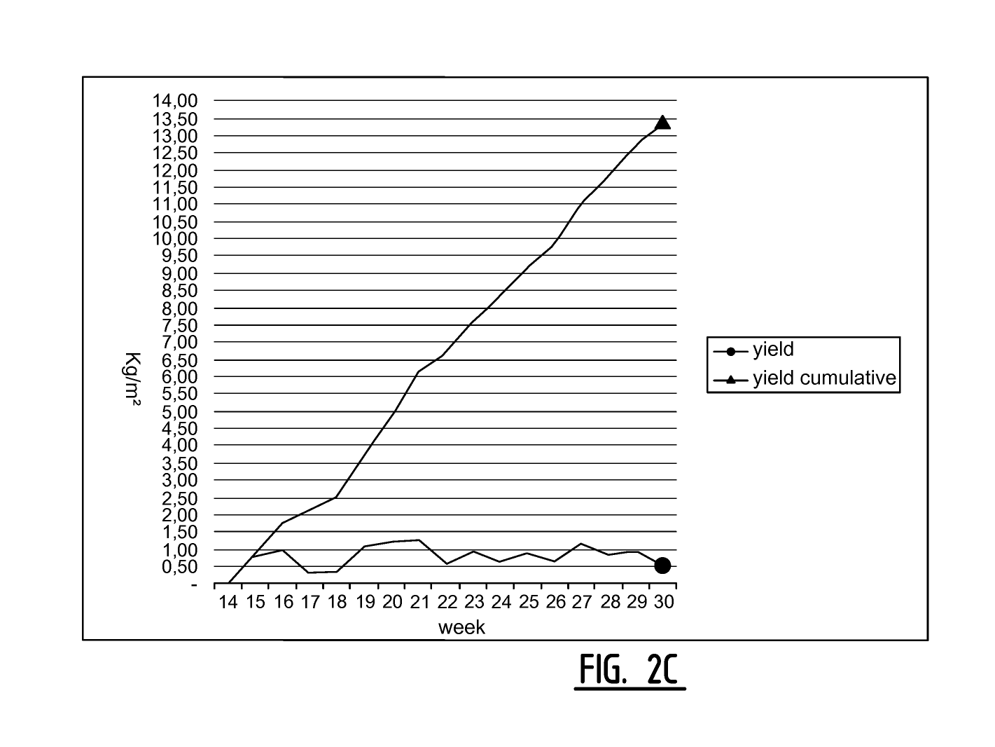

Peppers were harvested as normal, and every week the weight of the harvested peppers were determined. FIG. 2 shows that after 30 weeks, almost 13.5.0 kg/m² of peppers were harvested. This is about 90% of the yield obtained by the traditional cultivation of Marietta peppers, i.e. when watering the plants with water comprising mineral plant nutrition.

### EXAMPLE 3

### Organic Growing of Tomato Plants

Seeds of tomato plants of the variety Careza were sown in 100% coco substrate. Plants were cultivated as is common in the art, however, instead of watering with water comprising mineral plant nutrition the plants were watered with a mixture comprising the liquid having a high nitrate content of the present invention, basin water, Horti-organic A, Horti-organic B, micro nutrients, and iron. Two different mixture compositions were used throughout the experiment, starting with Mixture 1 and finishing with Mixture 2. The compositions of these mixtures are as indicated in Table 2. The mixture always comprised about 25% (volume) of the liquid having a high nitrate content of the present invention.

TABLE 2

| | |
|---|---|
| Mixture 1 | Mixture 2 |

| | | | | |
|---|---|---|---|---|
| Liquid having a | 125 | l | 150 | l |
| high nitrate | | | | |
| content of the | | | | |
| present invention | | | | |
| Basin water | 375 | l | 450 | l |
| Horti-organic A | 1.0 | l | 3.0 | l |
| Horti-organic B | 0.5 | l | 1.5 | l |
| Micro nutrients | 40.0 | ml | 50.0 | ml |
| Iron | 50.0 | g | 45.0 | g |

| | | | |
|---|---|---|---|
| Compost tea | 14.0 | l | — |

| | | | |
|---|---|---|---|
| Magnesium | — | 100 | g |
| sulphate | | | |

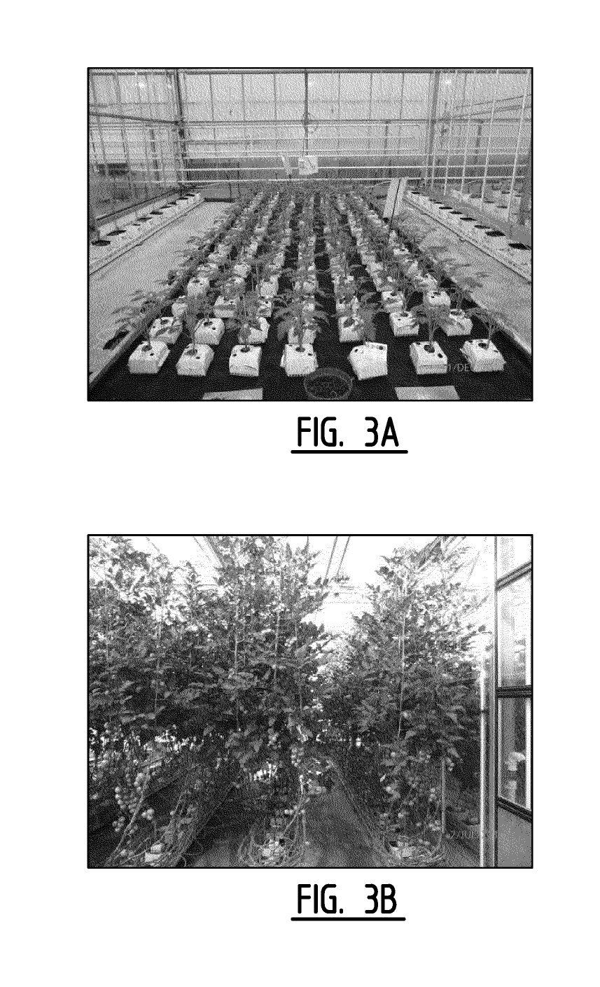

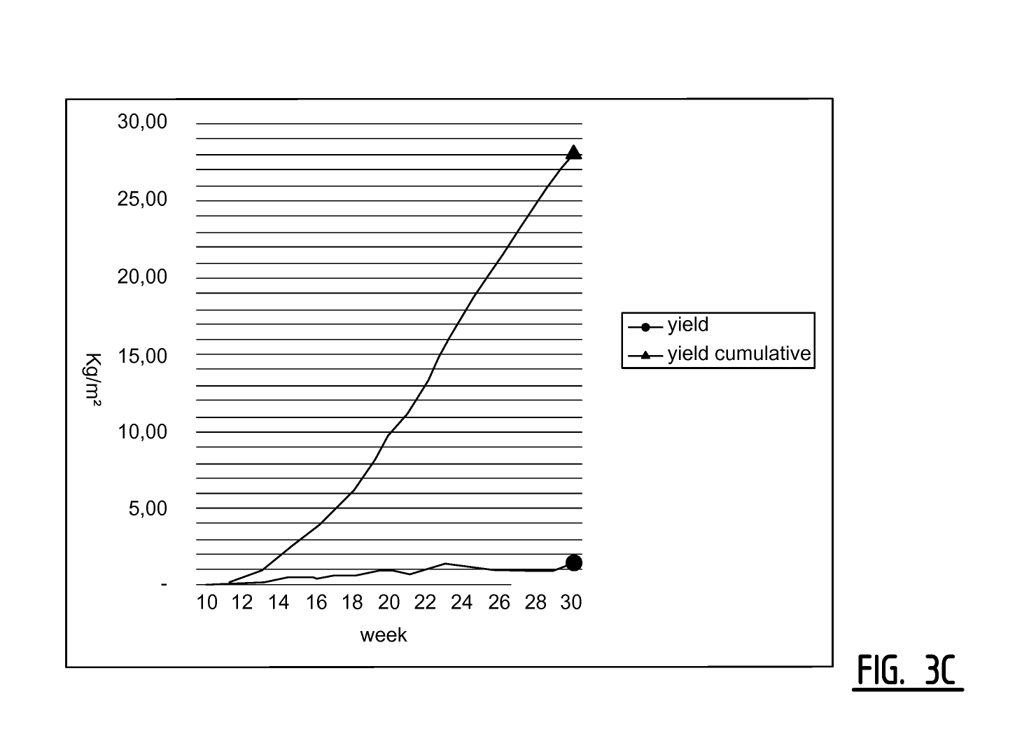

Tomato fruits were harvested as normal, and every week the weight of the harvested tomato fruits were determined. FIG. 3 shows that after 30 weeks, almost 27.0 kg/m² of tomato fruits were harvested. This is about 90% of the yield obtained by the traditional cultivation of Careza tomato plants, i.e. when watering the plants with water comprising mineral plant nutrition.

### EXAMPLE 4

### Organic Growing of Cucumber Plants

Seeds of cucumber plants of the variety Proloog are sown in 100% coco substrate. Plants are cultivated as is common in the art, however, instead of watering with water comprising mineral plant nutrition the plants are watered with a mixture comprising the liquid having a high nitrate content of the present invention, basin water, Horti-organic A, Horti-organic B, magnesium sulphate, micro nutrients, and iron, as indicated in Table 3.

TABLE 3

| | | |
|---|---|---|
| Liquid having a | 150 | l |
| high nitrate | | |
| content of the | | |
| present invention | | |
| Basin water | 350 | l |
| Horti-organic A | 3.0 | l |
| Horti-organic B | 1.5 | l |
| Micro nutrients | 50.0 | ml |
| Iron | 45.0 | g |
| Magnesium | 100 | g |
| sulphate | | |

Cucumber fruits are harvested as normal, and every week the weight of the harvested cucumber fruits are determined. About 90% of the yield obtained by the traditional cultivation of Proloog cucumber plants, i.e. when watering the plants with water comprising mineral plant nutrition, is achieved.

The results from these Examples indicate that the use of the organic liquid produced by the method of the present invention is commercially interesting for the organic market.

1. A method for producing an organic liquid having a high nitrate content, comprising:
a) contacting a solution comprising proteins and/or amino acids as a source of organic nitrogen and a liquid with ammonification bacteria to convert organic nitrogen into ammonium (NH₄⁺); and
b) contacting the solution with nitrifying bacteria to convert ammonium (NH₄⁺) to nitrate (NO₃⁻).

2. The method according to claim 1, wherein step b) takes place subsequently to step a).

3. The method according to claim 2, wherein steps a) and b) take place in separate containers.

4. The method according to claim 1, wherein step (a) is performed at a pH of above 8.0.

5. The method according to claim 1, wherein step (b) is performed at a pH of between 5.0 and 8.0.

6. The method according to claim 1, wherein the organic liquid has a nitrate content of at least 4 mmol/l.

7. The method according to claim 1, wherein the organic liquid having a high nitrate content is suitable for organically cultivating plants on a substrate.

8. An organic liquid having a high nitrate content obtainable by the method of claim 1.

9. An organic plant nutrition having a high nitrate content.

10. The organic plant nutrition according to claim 9 having a nitrate content of at least 4 mmol/l.

11. The organic plant nutrition according to claim 9 obtainable by the method comprising:
a) contacting a solution comprising proteins and/or amino acids as a source of organic nitrogen and a liquid with ammonification bacteria to convert organic nitrogen into ammonium (NH₄⁺); and
b) contacting the solution with nitrifying bacteria to convert ammonium (NH₄⁺) to nitrate (NO₃⁻).

12-14. (canceled)

15. A method for organically cultivating plants on a substrate, comprising adding an organic liquid having a high nitrate content to the substrate.

16. The method according to claim 15, wherein the organic liquid has a nitrate content of at least 4 mmol/l.

17. The method according to claim 15, wherein the organic liquid is prepared by the method comprising:
a) contacting a solution comprising proteins and/or amino acids as a source of organic nitrogen and a liquid with ammonification bacteria to convert organic nitrogen into ammonium (NH₄⁺); and
b) contacting the solution with nitrifying bacteria to convert ammonium (NH₄⁺) to nitrate (NO₃⁻).
